SEQUENCE LISTING (24 sequences)

SEQ ID NO: 1 - DNA, 1596 bp, Bacillus smithii
  CDS (1)..(1593): nadB
atggagaaagaagcggatgtagtgattatcgggggaggggtggcggctcttcagcttgctcgctatcttcatccgcaatttcatgtagtgatcatgacaaaaagcaaaatccgaaacagcaactcttacaaggcacagggagggattgctgcggttgtttccgagacggatcgattggatttgcatgttcaggatacgttagttgccggaaggtaccaccaatctactaaagtagtggatagcctcgtgcgggaaggttctgaagtggtgaaagagttaatcaaaagcggtcttcctgttgaccggaatgaaacgggagaggtggcgttaggacttgaaggagcccatagtcgccgccgaattgttcattgcggcggggatgcaacaggtcgttatttaattgaatttttattggatcggttaccgccaaatgttgaaatagtggagaatgagctggtttttgaactactgcttacaaacgatcgaagccgatgtatcggcgtagcggcaaaatcagttgacggttctgttcatcgctattttggccgtcatattgttctggcaacagggggagcaggcgcactttatccgtatacatccaatcatccgcgaatgatcggagatggagtggcgatggcttaccgagctggagcggcagtggctgatatggaatttgttcagttccacccaacattgttatacgtagatggagcaacaaaaggtttaatttcagaagcggtaagaggagccggagctattttactggacagtaaaggaaggcggatcatggaaggagttcatccacaaaaagatttggctccgagacatattgtcgcctatgagatttacaaaagaagagcagaacagcaggacgtttttttagatataagcggaatatctcattttgaagaaaagttcccgagcatttctcaattatgccgcgagcagggtgtggatcttaaggaaggaaaaatcccggtagtgcccggaagccactttttaatgggcggaatcgtcatcgattcctttggacgaacttcggtcaatggattatatgcgatcggggaaacagcatccagcggggtccatggagccaataggctggccagtaattctcttttagaagggcttgtgtttgggaaaagaacagctgagtttatcaaccatgaatctcacgtttcgtcgatacgatccgttctttatgactatccgttgaaagtgaaagaagacgaaatgccaaatctcgattcagaacagttgaaatggaacatgatcgaaaatgtaggcgtcatccgagatgaagccggacttaacagacaattgaattggctgccggttcagtttttggaatctttgatggaacaatctttggaaaggctatcaatcgaacaaattcaaacgatttttatgtttatcaatagttttatcatgacgaagtcggcattgattcggaaagagagtcgcggtgcccacatacgaagcgattttccaaatgaagaccttttttggaaaggtcggcgcattatccatgaacaaacgaaaataacatggaggagagggtcttatgaatcgcatcaaactggaatccatgcttaa

SEQ ID NO: 2 - protein, 531 aa, Bacillus smithii
MEKEADVVIIGGGVAALQLARYLHPQFHVVIMTKSKIRNSNSYKAQGGIAAVVSETDRLDLHVQDTLVAGRYHQSTKVVDSLVREGSEVVKELIKSGLPVDRNETGEVALGLEGAHSRRRIVHCGGDATGRYLIEFLLDRLPPNVEIVENELVFELLLTNDRSRCIGVAAKSVDGSVHRYFGRHIVLATGGAGALYPYTSNHPRMIGDGVAMAYRAGAAVADMEFVQFHPTLLYVDGATKGLISEAVRGAGAILLDSKGRRIMEGVHPQKDLAPRHIVAYEIYKRRAEQQDVFLDISGISHFEEKFPSISQLCREQGVDLKEGKIPVVPGSHFLMGGIVIDSFGRTSVNGLYAIGETASSGVHGANRLASNSLLEGLVFGKRTAEFINHESHVSSIRSVLYDYPLKVKEDEMPNLDSEQLKWNMIENVGVIRDEAGLNRQLNWLPVQFLESLMEQSLERLSIEQIQTIFMFINSFIMTKSALIRKESRGAHIRSDFPNEDLFWKGRRIIHEQTKITWRRGSYESHQTGIHA

SEQ ID NO: 3 - DNA, 1281 bp, Trichococcus flocculiformis
  CDS (1)..(1278): nadB
atgcgcaactatgatgtccttatcatcggaacgggggtcgccggactgtttgcagccctcaatctcagcagcgacaaacaaatcctgatcgccaccaaaggaactttggaagataacgattccttccttgcccaaggcggcatctgcgtccagcgcgacgaaatggatttcgagcctttcctggaggacactttgcgtgctggccactacgaaaataatgaagcggcagtcgcaaccatgatcaatcaatcacaagaaatcattgacgatctgatcgatctgggcgttgccttcgacaaaaagggagccggtttctcctataccaaagaaggcgctcactcccgagcccggatcctgcactgcaaagatatgaccggcaaggaaatcaacagcaaactgatcgcacgggtgaaagagctgaaaaatgtgagcatctttgagaacagcacgcttgtcgatcttttggttgccggcgaccgttgccatggcgcggtgctccgcgatcaggccggaaagttatccaacgtttatgcacagtccacgctgctcgccaccggtggcatcggcggcctgttcagcaaaacgaccaacttcgcacatttgacaggcgacgccatcgccatcgcgctgaagcacggtatccgcgtcaaggacctggattatgtccagatccatccgacttcgttgtataccggacgttcgggcagagctttcctgatgtcggaatcgctgcgtggggaaggcgccatcctcgtaggcaaagacggccaacgtttcgctgatgaactgttgccgcgtgacttgttgacgaaagcaatctacacccaaatggaaaaggatcgcttgccctatgtccggatgctcctgaaaggccacgtagaaggatcgatcgccggaaggtttccgggcatccatgcccactgcctgcaggaaggctatgatttggttgaagattacatcccggtcgtgcccgcccagcattatttcatgggcggcatcgcagtcgatctgaatagcaaaacttcgttggcgaacctttacgcagccggagaaacgagctgcaacggcgtccatgggaagaaccgcctggcgagcaactcgctgttggaaagtttggtattcagcaagcgcgccgcccaagccatcgatgagtcattccagtgcgcgcttccttatccgtttcccgaacctgatgcccgctccatcgaagcgggcctaaaggaagcaaacagaaaactattggaagaaaccggaagcgaggaggaagctcatgcaaagtaa

SEQ ID NO: 4 - protein, 426 aa, Trichococcus flocculiformis
MRNYDVLIIGTGVAGLFAALNLSSDKQILIATKGTLEDNDSFLAQGGICVQRDEMDFEPFLEDTLRAGHYENNEAAVATMINQSQEIIDDLIDLGVAFDKKGAGFSYTKEGAHSRARILHCKDMTGKEINSKLIARVKELKNVSIFENSTLVDLLVAGDRCHGAVLRDQAGKLSNVYAQSTLLATGGIGGLFSKTTNFAHLTGDAIAIALKHGIRVKDLDYVQIHPTSLYTGRSGRAFLMSESLRGEGAILVGKDGQRFADELLPRDLLTKAIYTQMEKDRLPYVRMLLKGHVEGSIAGRFPGIHAHCLQEGYDLVEDYIPVVPAQHYFMGGIAVDLNSKTSLANLYAAGETSCNGVHGKNRLASNSLLESLVFSKRAAQAIDESFQCALPYPFPEPDARSIEAGLKEANRKLLEETGSEEEAHAK

SEQ ID NO: 5 - protein, 534 aa, Pseudomonas putida
MSQQFQHDVLVIGSGAAGLSLALNLPSHLRVAVLSKGDLSNGSTFWAQGGVAAVLDNTDTVQSHVEDTLNAGGGLCHEDAVRFTVEHSREAIQWLIEQGVPFTRDEHYSVDDGGFEFHLTREGGHSHRRIIHAADATGAAIFTTLLEQARQRPNIQLLEQRVAVDLITERRLGLPGERCLGAYVLDRNTGEVDTFGARFTVLATGGAAKVYLYTSNPDGACGDGIAMAWRAGCRVANLEFNQFHPTCLYHPQAKSFLITEALRGEGALLRLPNGERFMPRFDPREELAPRDIVARAIDHEMKRLGVDCVYLDITHKPADFIKSHFPTVYERCLAFGIDITRQPIPVVPAAHYTCGGVMVDDCGHTDVPGLYAIGETSFTGLHGANRMASNSLLECFVYGRAAAADIQAHLEQVAMPKALPGWDASQVTDSDEDVIIAHNWDELRRFMWDYVGIVRTSKRLQRAQHRIRLLLDEIDEFYSNYKVSRDLIELRNLAQVAELMILSAMQRKESRGLHYTLDYPGMLDEAKDTILNPL

SEQ ID NO: 6 - protein, 540 aa, Escherichia coli
MNTLPEHSCDVLIIGSGAAGLSLALRLADQHQVIVLSKGPVTEGSTFYAQGGIAAVFDETDSIDSHVEDTLIAGAGICDRHAVEFVASNARSCVQWLIDQGVLFDTHIQPNGEESYHLTREGGHSHRRILHAADATGREVETTLVSKALNHPNIRVLERSNAVDLIVSDKIGLPGTRRVVGAWVWNRNKETVETCHAKAVVLATGGASKVYQYTTNPDISSGDGIAMAWRAGCRVANLEFNQFHPTALYHPQARNFLLTEALRGEGAYLKRPDGTRFMPDFDERGELAPRDIVARAIDHEMKRLGADCMFLDISHKPADFIRQHFPMIYEKLLGLGIDLTQEPVPIVPAAHYTCGGVMVDDHGRTDVEGLYAIGEVSYTGLHGANRMASNSLLECLVYGWSAAEDITRRMPYAHDISTLPPWDESRVENPDERVVIQHNWHELRLFMWDYVGIVRTTKRLERALRRITMLQQEIDEYYAHFRVSNNLLELRNLVQVAELIVRCAMMRKESRGLHFTLDYPELLTHSGPSILSPGNHYINR

SEQ ID NO: 7 - DNA, 1104 bp, Bacillus smithii
  CDS (1)..(1101): nadA
atgaatttgttgcaggcgcttcaacaaacaacggtgattccggagcattataacaagatgaccattcaagagatggaagaaagagtccgagaattgaaaactcgtttaggagaacgattgtttattcccgggcatcattatcaaagggatgaagtgattcaatttgcggatgccgttggagattcttttaagcttgcgaggatttccgctgataataaaaaggcggaatacatcgtcttttgcggtgttcattttatggcggagacggctgatattttgaccgcgcctgatcaaaaagtgatattgcccgataggaaagccggctgctcgatggcggatatggcggatatccatcaaacagagagggcatggaacaagctggcgaattggtttggcgacaccgttttgccattaacatatgtgaattccacggcggagatcaaagcatttgttggcagaaacggcggagccacggtcacttcttccaatgcccataaaatattgaaatggggattctcgcaaaaagaacgaattctgtttctcccggaccaacatcttggacgaaataccgcttatgatttaggtgtcgatcttcaacaaatggctctttgggacccgttgaaagacgaattgatctacgatggggatgtgaatgagattaaagtgattttatggaaagggcattgttctgtacatgaaaattttactatccataatgtccgggaagtacggaataaatatccgaacatcaatattatcgtacatccggaatgtcgtcgggaagtcgtggaggcttcggatagcagcggttccacgaactacattatccaaacaattgaaaaagcggcgccgggaacatcttgggcgattggaacagaaatgaatctcgtcagtcgtctgatcaaacaacatccggataaacatattatttcccttaacccgaatttttgcccgtgtatgacgatgaaccgtattgatcttccccatctgctttggtcattggagtccatttacaaaggagattccgtcaatttcatcaaggtgcctgaacaagtagcgattgaagccaaacttgcgcttgaacgaatgcttgcacttgcatga

SEQ ID NO: 8 - protein, 367 aa, Bacillus smithii
MNLLQALQQTTVIPEHYNKMTIQEMEERVRELKTRLGERLFIPGHHYQRDEVIQFADAVGDSFKLARISADNKKAEYIVFCGVHFMAETADILTAPDQKVILPDRKAGCSMADMADIHQTERAWNKLANWFGDTVLPLTYVNSTAEIKAFVGRNGGATVTSSNAHKILKWGFSQKERILFLPDQHLGRNTAYDLGVDLQQMALWDPLKDELIYDGDVNEIKVILWKGHCSVHENFTIHNVREVRNKYPNINIIVHPECRREVVEASDSSGSTNYIIQTIEKAAPGTSWAIGTEMNLVSRLIKQHPDKHIISLNPNFCPCMTMNRIDLPHLLWSLESIYKGDSVNFIKVPEQVAIEAKLALERMLALA

SEQ ID NO: 9 - protein, 285 aa, Trichococcus flocculiformis
MDELTERILQLKAEKDAVILAHYYAPGDVQDIADFIGDSYYLSKIFTAAQVTEKVIVFCGVAFMGESAKMLNPEKVILMPDLKADCPMAHMVKIRDIQKIRETYEDFTVAVVCYINSTAEIKAHADVCVTSSNAKNVIAALPNRYIYFIPDEHLGRYISHQLPEKTFFTLFNDGYCPIHTSIRVVDVLKMRRDLPQAEILAHPECKEEILDLADFVGSTSDLVAYTEKFTSLSQDFIICTEIGVIHDMEKRSPGKSFHAPSVGQVCPDMKLNTIENSKFAARTNP

SEQ ID NO: 10 - protein, 352 aa, Pseudomonas putida
MTQISERLLVQAHLDAKQPNPLTAEQEAEYRAAIAAELKAQNAVLVAHYYCDPVIQALAEETGGCVSDSLEMARFGKNHPAETVIVAGVRFMGETAKILTPEKRVLMPTLEATCSLDLGCPVEEFSAFCDQHPERTVVVYANTSAAVKARADWVVTSSCALEIVESLMDNGETIIWGPDQHLGRYIQKQTGADMLLWDGACIVHEEFKSRQLADMKALYPDAAILVHPESPEAVIELADAVGSTSQLIKAAQTLPNKTFIVATDRGIFYKMQQLCPDKEFVEAPTAGNGAACRSCAHCPWMAMNTLERVLDCLRNGTNEIFVDPALVPKAIKPLNRMLDFTQAARLKLSGNA

SEQ ID NO: 11 - protein, 347 aa, Escherichia coli
MSVMFDPDTAIYPFPPKPTPLSIDEKAYYREKIKRLLKERNAVMVAHYYTDPEIQQLAEETGGCISDSLEMARFGAKHPASTLLVAGVRFMGETAKILSPEKTILMPTLQAECSLDLGCPVEEFNAFCDAHPDRTVVVYANTSAAVKARADWVVTSSIAVELIDHLDSLGEKIIWAPDKHLGRYVQKQTGGDILCWQGACIVHDEFKTQALTRLQEEYPDAAILVHPESPQAIVDMADAVGSTSQLIAAAKTLPHQRLIVATDRGIFYKMQQAVPDKELLEAPTAGEGATCRSCAHCPWMAMNGLQAIAEALEQEGSNHEVHVDERLRERALVPLNRMLDFAATLRG

SEQ ID NO: 12 - DNA, 846 bp, Bacillus smithii
  CDS (1)..(843): nadC
atgaatcgcatcaaactggaatccatgcttaaagatttttttatcgaagatattggggacggggatataagtgcagaagccatttttacggctgatgatcgcggctctttttactttttgtcaaaggcggacggcgttttttgtggcagtgatgtcattaagacaggttttcgtctgatcgatcccacttcggaggtaaccgtatatcggaaagatggcgatgttgtggaaaaaggagaactcatagctgaaatcaagggtgcgatgcgcggccttctccaaggcgaaagggtgatcttaaatttgattcaaaggatgtccgggattgccacagtaacggctaaggcagttgaaatcgttaaagggacgggggcgaatatttgcgatacaagaaaaacaactcccggtctaaggatgcttgaaaaatatgccgttcgagtaggcggaggctataaccatcgcaaaggactttttgatgccattatgttaaaagataatcatattgcatttgcagggagtattttgtctgcggtgaaaagggcaaaacagtttgccgggcacacgacgaaagtagaagtggaaattgaaacgaaagaacagctgctggaagcggtagaagccggtgccgatgttattatgtttgataattgcactccggaacaaatacgcgattggatcatttatgtgccgaatcatattcgtacagaagcttcaggaggcattactttgtcgaatttgagagcgtatgccgaaacgggtgtacaatggatttctcttggctttttgacccactcttaccaagtgctcgatatcagcgcaaaggtgcagccaaattttatatcttaa

SEQ ID NO: 13 - protein, 281 aa, Bacillus smithii
MNRIKLESMLKDFFIEDIGDGDISAEAIFTADDRGSFYFLSKADGVFCGSDVIKTGFRLIDPTSEVTVYRKDGDVVEKGELIAEIKGAMRGLLQGERVILNLIQRMSGIATVTAKAVEIVKGTGANICDTRKTTPGLRMLEKYAVRVGGGYNHRKGLFDAIMLKDNHIAFAGSILSAVKRAKQFAGHTTKVEVEIETKEQLLEAVEAGADVIMFDNCTPEQIRDWIIYVPNHIRTEASGGITLSNLRAYAETGVQWISLGFLTHSYQVLDISAKVQPNFIS

SEQ ID NO: 14 - protein, 510 aa, Trichococcus flocculiformis
MSYDYNDDSLALHTDLYQINMMKTYWDEGITEKHAIFELYFRKYPFTFNNGYAVYAGLERFVSYIQNLRFTDTDIAYLREHVGYDEGFLEYLKNFRFSGTIRSALEFTGDLVFANEPIVQVEGPLAECQLIETALLNIVNFQTLIATKAARLRYVCNNEPLMEFGSRFTRAQEMDAAIWGTRAAYIAGFNATSNVRAAKMFGIPASGTHAHSMVQVYRNDYDAFMAYAFTHSHKDCVFLVDTYDTLKSGVPNAIKVAKEMGDKINFLGVRLDSGDMAYISKKVRQQLDEFTAGFTDAKIYASNDLDELTILNLKMQGAKIDVWGVGTKLITAYDQPALGAVYKLVSIADEFTNGQMVDTMKISSNAEKVSTPGKKQVWRITRKRDGKSEGDYIALWEEQPDQESELYMFHPFTVFTYINKTITDFEARPILQDIIVEGKLVYELPSLQEVKAFSEAQMSGLWDEYKRILNPEFTDYPVDLSQKTYDHKMATIKEIKKQIKEEASLLESTK

SEQ ID NO: 15 - protein, 282 aa, Pseudomonas putida
MPNLRLADLTAEIEANVRRALLEDIGSGDITAQLIPAERLAKATIITRENCVVAGTAWVDAVFRQLDPRVAVHWQVADGEQASANQPLFHLEGPARSLLSGERSALNFLQLLSGVATRARFLADLVAGTQVRLLDTRKTLPGLRLAQKYAVTCGGCDNHRIGLYDAFLIKENHIAASGGVAEAVAAAHRIAPGKPVEIEVESLDELRQALAAGADIIMLDELSLDEMREAVRITAGKAKLEASGGVNETTLRVIAETGVDYISIGAMTKDVKAVDLSMRLSL

SEQ ID NO: 16 - protein, 297 aa, Escherichia coli
MPPRRYNPDTRRDELLERINLDIPGAVAQALREDLGGTVDANNDITAKLLPENSRSHATVITRENGVFCGKRWVEEVFIQLAGDDVTIIWHVDDGDVINANQSLFELEGPSRVLLTGERTALNFVQTLSGVASKVRHYVELLEGTNTQLLDTRKTLPGLRSALKYAVLCGGGANHRLGLSDAFLIKENHIIASGSVRQAVEKASWLHPDAPVEVEVENLEELDEALKAGADIIMLDNFETEQMREAVKRTNGKALLEVSGNVTDKTLREFAETGVDFISVGALTKHVQALDLSMRFR

SEQ ID NO: 17 - DNA, 58 bp, Artificial Sequence
  E. coli BW25113 nadB isolation Forward primer
actggagctccaccccaggaaggaggaaaaaacatatgaatactctccctgaacattc

SEQ ID NO: 18 - DNA, 36 bp, Artificial Sequence
  E. coli BW25113 nadB isolation Reverse primer
actgggatccttatctgtttatgtaatgattgccgg

SEQ ID NO: 19 - DNA, 63 bp, Artificial Sequence
  P. putida KT2440 nadB isolation Forward primer
actggagctccaccccaggaaggaggaaaaaacatatgagccaacaattccaacatgatgtcc

SEQ ID NO: 20 - DNA, 32 bp, Artificial Sequence
  P. putida KT2440 nadB isolation Reverse primer
actgggatcctcagagcgggttaaggatggtg

SEQ ID NO: 21 - DNA, 26 bp, Artificial Sequence
  B. smithii DSM 4216 nadB isolation Forward primer
cagtcatatggagaaagaagcggatg

SEQ ID NO: 22 - DNA, 31 bp, Artificial Sequence
  B. smithii DSM 4216 nadB isolation Reverse primer
cagtggatccttaagcatggattccagtttg

SEQ ID NO: 23 - DNA, 25 bp, Artificial Sequence
  T. flocculiformis DSM 2094 nadB isolation Forward primer
catatgatgcgcaactatgatgtcc

SEQ ID NO: 24 - DNA, 27 bp, Artificial Sequence
  T. flocculiformis DSM 2094 nadB isolation Reverse primer
ggatccttactttgcatgagcttcctc

(19) United States
(12) Patent Application Publication
(10) Pub. No.: US 2022/0010266 A1
(43) Pub. Date: Jan. 13, 2022

(54) Microbial hosts engineered for increased tolerance to temperature shifts

(71) Applicant: Wageningen Universiteit, Wageningen (NL)

(72) Inventors: Linde Francisca Cornelia Kampers, Wageningen (NL); Joanna Maria Volkers, Wageningen (NL); Petrus Johannes Schaap, Ede (NL); Vitor Alexandre Pires Martins dos Santos, Wolfenbuettel (DE)

(73) Assignee: Wageningen Universiteit, Wageningen (NL)

(21) Appl. No.: 17/291,620

(22) PCT Filed: Nov. 8, 2019
(86) PCT No.: PCT/EP2019/080645

§ 371 Date: May 6, 2021

(30) Foreign Application Priority Data
Nov. 9, 2018 (EP) 18205369.4

(51) Int. Cl. C12N 1/20; C12N 9/06; C12N 9/10

(52) U.S. Cl. CPC C12N 1/20; C12N 9/0022; C12N 9/1048; C12Y 205/01072; C12Y 104/03016; C12Y 204/02019; C12N 9/1085

(57) ABSTRACT

The present invention relates to microbial host cells that have been engineered for increased tolerance to temperature shifts, for increased performance at temperatures different from the microorganism's optimal temperature and/or for changing at least one of the microorganism's cardinal temperatures by replacing an endogenous NAD⁺ biosynthesis gene by a heterologous gene encoding a corresponding enzyme with another temperature profile and/or from a microorganism with a different optimum growth temperature. The invention further relates to processes wherein the engineered microbial host cells are used for producing a fermentation product, and to the use nucleotide sequences encoding NAD⁺ biosynthesis gene for changing at least one of a microorganism's cardinal temperatures and/or for improving a microorganism's tolerance to temperature shifts.

### FIELD OF THE INVENTION

The present invention relates to the field of molecular microbiology, metabolic engineering, synthetic biology and fermentation technology. In particular, the invention relates to microbial host cells that have been engineered for increased tolerance to temperature shifts, for increased performance at temperatures different from the microorganism's optimal temperature and/or for changing at least one of the microorganism's cardinal temperatures by replacing an endogenous NAD⁺ biosynthesis gene by a heterologous gene encoding a corresponding enzyme with another temperature profile.

### BACKGROUND ART

Mesophiles are the preferred organisms for industry. They are genetically accessible, easy to culture, and have established methods to adapt the metabolism for optimization of production. However, it is often beneficial to use bacteria which are robust against a broad temperature range. Fermentation at high temperature offers several advantages as compared to mesophilic microorganisms, including higher growth and metabolic rates, lower cellular growth yield, increased physicochemical stability of enzymes and organisms and facilitated reactant activity and product recovery [1], [2]. Downsides to use thermophiles include the genetic inaccessibility, but also the high costs that are associated to culturing only at such high temperatures. To adapt a mesophile to have more resilience to higher temperatures would provide the benefits of both.

This would open up many different applications for mesophilic industrial workhorses, including increasing the production yield, robustness of the production process, the possibility of inducement with temperature shifts, or applying thermophilic new enzymes that are much more stable than their counterparts from mesophiles [3, 4].

Nicotinamide Adenine Dinucleotide (NAD⁺) is a cofactor essential for survival in all living organisms, by balancing the redox balance. In prokaryotes, de novo biosynthesis of NAD⁺ proceeds via a condensation reaction of L-aspartate and dihydroxyacetone phosphate, catalysed by the quinolinate synthase system [5]. This proposed complex is composed of two enzymes: L-aspartate oxidase (NadB) which catalyses the oxidation of L-aspartate to iminoaspartate using O₂ as an electron receptor, releasing H₂O₂, and quinolinate synthase (NadA), which condenses iminoaspartate with dihydroxyacetone phosphate to produce quinolinate.

It is an object of the present invention to provide for microbial host cells that have been engineered for increased tolerance to temperature shifts and/or for increased performance at temperatures different from their strain specific optimal temperature.

### SUMMARY OF THE INVENTION

In a first aspect, the invention pertains to a microbial host cell comprising a nucleotide sequence encoding a heterologous NAD⁺ biosynthesis enzyme, wherein at least one of: a) the heterologous NAD⁺ biosynthesis enzyme is from a microbial donor organism with an optimum growth temperature that is different from the optimum growth temperature of the microbial host cell, or from a microbial donor organism that has a wider range of growth temperatures than the microbial host cell; and, b) the heterologous NAD⁺ biosynthesis enzyme has a higher activity than the corresponding endogenous NAD⁺ biosynthesis enzyme of the host cell at a temperature that differs from the optimum growth temperature of the host cell, as determined in an assay for activity of the NAD⁺ biosynthesis enzyme wherein the activity of the endogenous and heterologous NAD⁺ biosynthesis enzymes is determined over a period of time of at least 10 minutes. Preferably, the heterologous NAD⁺ biosynthesis enzyme encoded by the nucleotide sequence comprised in the microbial host cell is selected from the group consisting of L-aspartate oxidase, quinolinate synthase and quinolinate phosphoribosyl-transferase, and wherein preferably the microbial host cell comprises nucleotide sequences encoding two or all three of the NAD⁺ biosynthesis enzyme from the group consisting of L-aspartate oxidase, quinolinate synthase and quinolinate phosphoribosyl-transferase. A preferred microbial host cell according to the invention is a host cell wherein the temperature difference in at least one of a) and b) above, is at least 2° C. More preferably, a microbial host cell according to the invention is a host cell, wherein at least one of: a) the heterologous NAD⁺ biosynthesis enzyme has a higher activity than the corresponding endogenous NAD⁺ biosynthesis enzyme in the host cell at a temperature that is higher than the optimum growth temperature of the host cell; and, b) the heterologous NAD⁺ biosynthesis enzyme is from a microbial donor organism with an optimum growth temperature that is higher than the optimum growth temperature of the microbial host cell.

In one embodiment, a microbial host cell according to the invention comprises a genetic modification that reduces or eliminates the specific activity of an endogenous NAD⁺ biosynthesis enzyme that corresponds to the heterologous NAD⁺ biosynthesis enzyme encoded by the nucleotide sequence comprised in the host cell, wherein preferably, the nucleotide sequence encoding a heterologous NAD⁺ biosynthesis enzyme replaces the endogenous nucleotide sequence encoding the corresponding endogenous NAD⁺ biosynthesis enzyme.

A microbial host cell according to the invention preferably is a yeast, a filamentous fungus, a eubacterium or an archaebacterium, more preferably the host cell is a Gram-positive or a Gram-negative bacterium. A microbial host cell according to the invention can e.g. be a host cell of a genus selected from the group consisting of: Escherichia, Anabaena, Actinomyces, Acetobacter, Caulobacter, Clostridium, Gluconobacter, Gluconacetobacter, Rhodobacter, Pseudomonas, Paracoccus, Bacillus, Brevibacterium, Corynebacterium, Rhizobium Sinorhizobium, Flavobacterium, Klebsiella, Enterobacter, Lactobacillus, Lactococcus, Streptococcus, Oenococcus, Leuconostoc, Pediococcus, Carnobacterium, Propionibacterium, Enterococcus, Bifidobacterium, Methylobacterium, Micrococcus, Staphylococcus, Streptomyces. Zymomonas, Streptococcus, Bacteroides, Selenomonas, Megasphaera, Burkholderia, Cupriavidus, Ralstonia, Methylobacterium, Methylovorus, Rhodopseudomonas, Acidiphilium, Dinoroseobacter, Agrobacterium, Sulfolobus, Sphingomonas, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, Ustilago, Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, and Sporobolomyces.

The heterologous NAD⁺ biosynthesis enzyme encoded by the nucleotide sequence that is comprised in a microbial host cell of the invention, preferably is an NAD⁺ biosynthesis enzyme that is obtained or obtainable from a microbial donor organism, which is a psychrophilic, a psychrotrophic or a thermophilic organism. In a preferred embodiment, the microbial host cell is a mesophile.

In one embodiment of a microbial host cell according to the invention, the heterologous NAD⁺ biosynthesis enzyme is a modified version of an enzyme that is endogenous to the host cell, which modified version enzyme comprises at least one modification in its amino acid sequence as compared to the endogenous enzyme, and wherein the modified version has a higher activity than the endogenous enzyme at a temperature that differs from the optimum growth temperature of the host cell, in an assay for activity of the NAD⁺ biosynthesis enzyme wherein the activity of the endogenous and the modified enzymes is determined over a period of time of at least 10 minutes.

The heterologous NAD⁺ biosynthesis enzyme encoded by the nucleotide sequence that is comprised in a microbial host cell of the invention, preferably is an NAD⁺ biosynthesis enzyme comprising an amino acid sequence selected from the group consisting of: a) an amino acid sequence that is at least 45% identical to SEQ ID NO: 2; b) an amino acid sequence that is at least 45% identical to SEQ ID NO: 4; c) an amino acid sequence that is at least 45% identical to SEQ ID NO: 5; d) an amino acid sequence that is at least 45% identical to SEQ ID NO: 6; e) an amino acid sequence that is at least 45% identical to SEQ ID NO: 8; f) an amino acid sequence that is at least 45% identical to SEQ ID NO: 9; g) an amino acid sequence that is at least 45% identical to SEQ ID NO: 10; h) an amino acid sequence that is at least 45% identical to SEQ ID NO: 11; i) an amino acid sequence that is at least 45% identical to SEQ ID NO: 13; j) an amino acid sequence that is at least 45% identical to SEQ ID NO: 14; k) an amino acid sequence that is at least 45% identical to SEQ ID NO: 15; and, I) an amino acid sequence that is at least 45% identical to SEQ ID NO: 16.

In a second aspect, the invention relates to a process for producing a fermentation product, the process comprises the steps of: (a) culturing a microbial host cell of the invention in a medium, whereby the host cell converts nutrients in the medium to the fermentation product; and, (b) optionally, recovery of the fermentation product. Preferably, the process comprises a shift in temperature, wherein preferably the shift in temperature is a shift of at least 2, 5, 7 or 10° C.

In a third aspect, the invention relates to the use of a nucleotide sequence encoding a NAD⁺ biosynthesis enzyme that is heterologous to a microbial host cell, wherein the nucleotide sequence is used for at least one of: a) changing at least one of the minimum, maximum and optimum growth temperature of the microbial host cell; and, b) improving resistance of the microbial host cell to a shift in temperature, wherein preferably the resistance of the microbial host cell to a shift to a higher temperature is improved. Preferably, in the use of a nucleotide sequence encoding a NAD+ biosynthesis enzyme that is heterologous to a microbial host cell, at least one of the microbial host cell and the nucleotide sequence encoding a heterologous NAD⁺ biosynthesis enzyme is as defined herein. Preferably in this aspect, at least one of: a) at least one of the minimum, maximum and optimum growth temperature of the microbial host cell is changed by at least 1° C.; and, b) the lag phase of the microbial host cell upon a shift in temperature of at least 2° C., is reduced by at least a factor 1.1.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention. Indeed, the present invention is in no way limited to the methods and materials described.

For purposes of the present invention, the following terms are defined below.

As used herein, the term “and/or” indicates that one or more of the stated cases may occur, alone or in combination with at least one of the stated cases, up to with all of the stated cases.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (polynucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWlN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly(G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative Conservative Amino Acid Residue Substitution Classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative Physical and Functional Classifications of Amino Acid Residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P |
| | and T |
| Flexible residues | Q, T, K, S, G, P, D, E, and R |

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3rd edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. An inducible promoter may also be present but not induced.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed earlier herein.

The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous and exogenous nucleic acids or proteins are not endogenous to the cell into which it is introduced, but have been obtained from another cell or synthetically or recombinantly produced. Generally, though not necessarily, such nucleic acids encode proteins, i.e. exogenous proteins, that are not normally produced by the cell in which the DNA is transcribed or expressed. Similarly exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other. The terms heterologous and exogenous specifically also apply to non-naturally occurring modified versions of otherwise endogenous nucleic acids or proteins.

The “specific activity” of an enzyme is herein understood to mean the amount of activity of a particular enzyme per amount of total host cell protein, usually expressed in units of enzyme activity per mg total host cell protein. In the context of the present invention, the specific activity of a particular enzyme may be increased or decreased as compared to the specific activity of that enzyme in an (otherwise identical) wild type host cell.

The term “fermentation” or “fermentation process” is herein broadly defined in accordance with its common definition as used in industry as any (large-scale) microbial process occurring in the presence or absence of oxygen, comprising the cultivation of at least one microorganism whereby preferably the microorganism produces a useful product at the expense of consuming one or more organic substrates. The term “fermentation” is herein thus has a much broader definition than the more strict scientific definition wherein it is defined as being limited a microbial process wherein the microorganism extracts energy from carbohydrates in the absence of oxygen. Likewise, the term “fermentation product” is herein broadly defined as any useful product produced in a (large-scale) microbial process occurring in the presence or absence of oxygen.

### DETAILED DESCRIPTION OF THE INVENTION

The present inventors surprisingly found that directing the redox balance in mesophilic strains by replacement of innate genes in the biosynthesis of NAD⁺ for genes from thermophiles or psychrophiles has a direct and clear effect on the robustness of the strain against large shifts in temperature. The nadB gene was knocked out in the two mesophilic species Escherichia coli and Pseudomonas putida, after which the nadB gene of the thermophile Bacillus smithii or the psychrotolerant mesophile Trichococcus flocculiformis was introduced. As a result, the lag phase in E. coli after a temperature shift from 3° C. to 44° C. was shortened by a factor 10. In P. putida, a temperature shift from 30° C. to 40° C. caused an increase in maximum growth rate of a factor 2. The present invention therefore provides methods and means for engineering microbial host cells for increased tolerance to temperature shifts, for increased performance at temperatures different from their strain specific optimal temperature and/or for changing at least one of the strain's cardinal temperatures.

In a first aspect therefore the invention pertains to a microbial host cell comprising a nucleotide sequence encoding a heterologous NAD⁺ biosynthesis enzyme. Preferably, the heterologous NAD⁺ biosynthesis enzyme encoded by the nucleotide sequence is characterized by at least one of the features: a) the heterologous NAD⁺ biosynthesis enzyme is from a microbial donor organism with an optimum growth temperature that is different from the optimum growth temperature of the microbial host cell, or from a microbial donor organism that has a wider range of growth temperatures than the microbial host cell; and, b) the heterologous NAD⁺ biosynthesis enzyme has a higher activity than the corresponding endogenous NAD⁺ biosynthesis enzyme of the host cell at a temperature that differs from the optimum growth temperature of the host cell, as determined in an assay for activity of the NAD⁺ biosynthesis enzyme, and wherein preferably the activity of the endogenous and the heterologous NAD⁺ biosynthesis enzymes is determined over a period of time of at least 5, 10, 20, 30 or 60 minutes. Preferably, the temperature difference in at least one of a) and b) is at least 2, 5, 10, 20, 30, 40, 60 or 80° C.

Methods for determining the optimum growth temperature of a microorganism are known in the art per se (see e.g. Laboratory Methods in Food Microbiology by W. F. Harrigan, Gulf Professional Publishing, 1998). The optimal temperature of a microorganism is herein defined as given in the Dictionary for Microbiology (Jacobs, M. B., M. J. Gerstein, and W. G. Walter. 1957. Dictionary of microbiology. Van Nostrand, New York).

Methods for determining the enzymatic activity of NAD⁺ biosynthesis enzymes are also known in the art per se. Suitable method for determining e.g. the activity of L-aspartate oxidase, quinolinate synthase and quinolinate phosphoribosyl-transferase are e.g. described in [4, 6].

Regarding the effects of temperature on the rate of reactions catalysed by enzymes it is generally known (see e.g. http://en.wikipedia.org/wiki/Enzyme_assay) that all enzymes work within a range of temperature specific to the organism. Increases in temperature generally lead to increases in reaction rates. There is however a limit to the increase because higher temperatures lead to a sharp decrease in reaction rates, which is due to the denaturation of the enzyme's three-dimensional structure which renders the enzyme inactive. The perceived “optimum” temperature for human enzymes is usually between 35 and 40° C. as the average body temperature for humans is 37° C. Human enzymes start to denature quickly at temperatures above 40° C. In contrast, enzymes from thermophilic archaea found in the hot springs are stable up to 100° C. However, the idea of an “optimum” rate of an enzyme reaction is misleading, as the rate observed at any temperature is the product of two rates, the reaction rate and the denaturation rate. If one was to use an assay measuring activity for one second, it would give high activity at high temperatures, however if one was to use an assay measuring product formation over an hour, it would give low activity at these temperatures. For this reason, feature b) above requires that the comparison of the activities of the endogenous and the heterologous NAD⁺ biosynthesis enzymes at different temperatures is determined over a minimum period of time of at least 5, 10, 20, 30 or 60 minutes so that the assay takes proper account of the effects of temperature on the enzyme's stability.

In one embodiment, the heterologous NAD⁺ biosynthesis enzyme is from a microbial donor organism that has an optimum growth temperature that is higher than the optimum growth temperature of the microbial host cell. Preferably, the optimum growth temperature of the microbial donor organism is at least 2, 5, 10, 15, 20, 25, 30, 35, 40, 50, 60 or 80° C. higher than the optimum growth temperature of the microbial host cell. Preferably therefore, when the microbial host cell is a psychrophilic or psychrotrophic organism, the microbial donor organism can be a mesophilic or thermophilic organism. Preferably therefore, when the microbial host cell is a mesophilic organism, the microbial donor organism can be a thermophilic organism. The thermophilic donor organism can be a moderate (simple) thermophile, an extreme thermophile or a hyperthermophile.

A psychrophile or cryophile, i.e. psychrophilic or cryophilic organism is herein defined as an organism having an optimal temperature for growth at about 15° C. or lower, a maximal temperature for growth at about 20° C., and a minimal temperature for growth at 0° C. [7] They are found in places that are permanently cold, such as the polar regions and the deep sea. Suitable psychrophilic organisms for use as donor or host organism in the present invention are e.g. Chryseobacterium antarcticum, Flavobacterium antarcticum, Pseudomonas fragi, Rhodococcus erythropolis and Bacillus simplex [8].

A psychrotrophic organism is capable of surviving or even thriving in extremely cold environment and thus also has a minimal temperature for growth at 0° C. or below like a psychrophile. However, the maximum and optimum growth temperatures of psychrotrophic organism are higher than that of a psychrophile. An example of a psychrotrophic organism for use as donor or host organism in the present invention is Trichococcus flocculiformis (e.g. strain DSM2094), which is a psychrotolerant mesophile, with an optimal growth temperature of 35° C., temperature growth range of 2-40° C. Other examples of psychrotrophes are Pseudomonas fluorescens, Serratia marcescens, Klebsiella oxytoca, Bacillus subtilis, Bacillus cereus and Paenibacillus polymyxa [9].

A thermophile is herein defined as an organism that thrives at relatively high temperatures, between 41 and 122° C. Thermophiles can be classified according to their optimal growth temperatures as a moderate (or simple) thermophile (50-64° C.), an extreme thermophile (65-79° C.) and a hyperthermophile (80° C. and beyond). Suitable thermophilic organism for use as donor or host organism in the present invention are e.g. Bacillus smithii, Methanobacterium the rmoautotrophicus, Clostridium thermocellum, Clostridium thermohydrosulfuricum and Sulfolobus tokodaii [1-3].

In one embodiment, the heterologous NAD⁺ biosynthesis enzyme is from a microbial donor organism that has an optimum growth temperature that is lower than the optimum growth temperature of the microbial host cell. Preferably, the optimum growth temperature of the microbial donor organism is at least 2, 5, 10, 15, 20, 25, 30, 35, 40, 50, 60 or 80° C. lower than the optimum growth temperature of the microbial host cell.

Preferably therefore, when the microbial host cell is a thermophilic organism (e.g. a moderate (simple) thermophile, an extreme thermophile or a hyperthermophile), the microbial donor organism can be a mesophilic, psychrophilic or psychrotrophic organism. Preferably therefore, when the microbial host cell is a mesophilic organism, the microbial donor organism can be a psychrophilic or psychrotrophic organism.

In one embodiment, the heterologous NAD⁺ biosynthesis enzyme is from a microbial donor organism that has a range of growth temperatures that has a higher maximal growth temperature than the range of growth temperatures of the microbial host cell. Preferably, the maximal growth temperature of the range of growth temperatures of the microbial donor organism is at least 2, 5, 10, 15, 20, 25, 30, 35, 40, 50, 60 or 80° C. higher than the maximal growth temperature of the range of the microbial host cell.

In one embodiment, the heterologous NAD⁺ biosynthesis enzyme is from a microbial donor organism that has a range of growth temperatures that has a lower minimal growth temperature than the range of growth temperatures of the microbial host cell. Preferably, the minimal growth temperature of the range of growth temperatures of the microbial donor organism is at least 2, 5, 10, 15, 20, 25, 30, 35, 40, 50, 60 or 80° C. lower than the minimal growth temperature of the range of the microbial host cell.

In one embodiment, the heterologous NAD⁺ biosynthesis enzyme is from a microbial donor organism that has a range of growth temperatures that has both a higher maximal growth temperature than the range of growth temperatures of the microbial host cell, and a lower minimal growth temperature than the range of growth temperatures of the microbial host cell. Preferably, the maximal growth temperature of the range of growth temperatures of the microbial donor organism is at least 2, 5, 10, 15, 20, 25, 30, 35, 40, 50, 60 or 80° C. higher than the maximal growth temperature of the range of the microbial host cell, and the minimal growth temperature of the range of growth temperatures of the microbial donor organism is at least 2, 5, 10, 15, 20, 25, 30, 35, 40, 50, 60 or 80° C. lower than the minimal growth temperature of the range of the microbial host cell.

In one embodiment, the heterologous NAD⁺ biosynthesis enzyme has a higher activity than the corresponding endogenous NAD⁺ biosynthesis enzyme of the host cell at a temperature that is higher than the optimum growth temperature of the host cell, as determined in an assay for activity of the NAD⁺ biosynthesis enzyme, and wherein preferably the activity of the endogenous and the heterologous NAD⁺ biosynthesis enzymes is determined over a period of time of at least 5, 10, 20, 30 or 60 minutes. Preferably, the heterologous NAD⁺ biosynthesis enzyme has an activity that is at least 10% higher than the activity of the endogenous enzyme at a temperature that is at least 2, 5, 10, 15, 20, 25, 30, 35, 40, 50, 60 or 80° C. higher than the optimum growth temperature of the host cell, when the activity of the endogenous and the heterologous NAD⁺ biosynthesis enzymes is determined in the assay for at least 20 minutes. More preferably, the heterologous NAD⁺ biosynthesis enzyme has an activity that is at least 20% higher than the activity of the endogenous enzyme at a temperature that is at least 2, 5, 10, 15, 20, 25, 30, 35, 40, 50, 60 or 80° C. higher than the optimum growth temperature of the host cell, when the activity of the endogenous and the heterologous NAD⁺ biosynthesis enzymes is determined in the assay for at least 20 minutes. Most preferably, the heterologous NAD⁺ biosynthesis enzyme has an activity that is at least 50% higher than the activity of the endogenous enzyme at a temperature that is at least 2, 5, 10, 15, 20, 25, 30, 35, 40, 50, 60 or 80° C. higher than the optimum growth temperature of the host cell, when the activity of the endogenous and the heterologous NAD⁺ biosynthesis enzymes is determined in the assay for at least 20 minutes.

In one embodiment, the heterologous NAD⁺ biosynthesis enzyme has a higher activity than the corresponding endogenous NAD⁺ biosynthesis enzyme of the host cell at a temperature that is lower than the optimum growth temperature of the host cell, as determined in an assay for activity of the NAD⁺ biosynthesis enzyme, and wherein preferably the activity of the endogenous and the heterologous NAD⁺ biosynthesis enzymes is determined over a period of time of at least 5, 10, 20, 30 or 60 minutes. Preferably, the heterologous NAD⁺ biosynthesis enzyme has an activity that is at least 10% higher than the activity of the endogenous enzyme at a temperature that is at least 2, 5, 10, 15, 20, 25, 30, 35, 40, 50, 60 or 80° C. lower than the optimum growth temperature of the host cell, when the activity of the endogenous and the heterologous NAD⁺ biosynthesis enzymes is determined in the assay for at least 20 minutes. More preferably, the heterologous NAD⁺ biosynthesis enzyme has an activity that is at least 20% higher than the activity of the endogenous enzyme at a temperature that is at least 2, 5, 10, 15, 20, 25, 30, 35, 40, 50, 60 or 80° C. lower than the optimum growth temperature of the host cell, when the activity of the endogenous and the heterologous NAD⁺ biosynthesis enzymes is determined in the assay for at least 20 minutes. Most preferably, the heterologous NAD⁺ biosynthesis enzyme has an activity that is at least 50% higher than the activity of the endogenous enzyme at a temperature that is at least 2, 5, 10, 15, 20, 25, 30, 35, 40, 50, 60 or 80° C. lower than the optimum growth temperature of the host cell, when the activity of the endogenous and the heterologous NAD⁺ biosynthesis enzymes is determined in the assay for at least 20 minutes.

It is herein understood that an NAD⁺ biosynthesis enzyme that is heterologous to the microbial host cell of the invention not only includes enzymes that are from a different species than the host cell but also includes modified versions of enzymes that are endogenous to the host cell but that comprise one or more amino acid modifications, preferably modifications that alter the temperature profile of the enzyme's activity. Therefore, in one embodiment, a host cell of the invention comprises a nucleotide sequence encoding a heterologous NAD⁺ biosynthesis enzyme, wherein the heterologous NAD⁺ biosynthesis enzyme is a modified version of an enzyme that is endogenous to the host cell. A modified version of an endogenous enzyme is herein understood to comprise at least one modification in its amino acid sequence as compared to the endogenous enzyme. Such a modification of the amino acid sequence can be at least one of a substitution, an insertion or a deletion of one or more amino acids as compared to the amino acid sequence of the wild type endogenous enzyme. Preferably, the modified version has a higher activity than the endogenous enzyme at a temperature that differs from the optimum growth temperature of the host cell, in an assay for activity of the NAD⁺ biosynthesis enzyme wherein the activity of the endogenous and the modified enzymes is determined over a period of time of at least 5, 10, 20, 30 or 60 minutes.

In a host cell of the invention, a nucleotide sequence encoding a heterologous NAD⁺ biosynthesis enzyme is preferably comprised in an expression construct, wherein the coding nucleotide sequence is operably linked to expression regulatory sequences that are capable of effecting expression of the coding nucleotide sequence in the host cell. A preferred host cell of the invention thus expresses or is at least capable of expressing a heterologous NAD⁺ biosynthesis enzyme.

To increase the likelihood that a heterologous NAD⁺ biosynthesis enzyme is expressed at sufficient levels and in active form in the transformed host cells of the invention, a nucleotide sequence encoding such heterologous NAD⁺ biosynthesis enzyme of the invention, is preferably adapted to optimize their codon usage to that of the host cell in question. The adaptiveness of a nucleotide sequence encoding an enzyme to the codon usage of a host cell may be expressed as codon adaptation index (CAI). The codon adaptation index is herein defined as a measurement of the relative adaptiveness of the codon usage of a gene towards the codon usage of highly expressed genes in a particular host cell or organism. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987, Nucleic Acids Research 15: 1281-1295; also see: Jansen et al, 2003, Nucleic Acids Res. 3J_(8):2242-51). An adapted nucleotide sequence preferably has a CAI of at least 0.2, 0.3, 0.4, 0.5, 0.6, 0.7, 0.8 or 0.9.

A heterologous NAD⁺ biosynthesis enzyme comprised or to be expressed in the host cell of the invention can be an enzyme from an NAD⁺ salvage pathway, wherein the cells salvage preformed precursors containing a pyridine base, such as e.g. nicotinic acid, nicotinamide and/or nicotinamide riboside, to form NAD⁺. Preferably however, the heterologous NAD⁺ biosynthesis enzyme comprised or to be expressed in the host cell of the invention is an enzyme from a de novo NAD⁺ biosynthesis pathway, wherein NAD⁺ is synthesized de novo from quinolinic acid that is generated from either tryptophan or aspartic acid. In a preferred embodiment the heterologous NAD⁺ biosynthesis enzyme is selected from the group consisting of L-aspartate oxidase, quinolinate synthase and quinolinate phosphoribosyl-transferase.

L-aspartate oxidase (EC 1.4.3.16) is herein understood as an enzyme that catalyses the reaction: L-aspartate+O₂↔H iminosuccinate+H₂O₂. L-aspartate oxidase is a flavoprotein (FAD) and is also known as quinolinate synthase B, indicated as AO, NadB or LASPO in literature. An L-aspartate oxidase for use as heterologous NAD⁺ biosynthesis enzyme is further preferably defined as comprising an amino acid sequence that is at least 45, 50, 55, 60, 65, 70, 75, 80, 85, 90, 95, 98, 99 or 100% identical to at least one of SEQ ID NO.'s: 2, 4, 5 and 6, or a nadB gene encoding such amino acid sequences.

Quinolinate synthase (EC 2.5.1.72) is herein understood as an enzyme that catalyses the reaction: glycerone phosphate (di hydroxyacetone phosphate)+iminosuccinate (iminoaspartate)↔H pyridine-2,3-dicarboxylate+2H₂O+phosphate. Quinolinate synthase is an iron-sulfur protein that requires a [4Fe-4S] cluster for activity and is either indicated as NadA or QS in literature. A quinolinate synthase for use as heterologous NAD⁺ biosynthesis enzyme is further preferably defined as comprising an amino acid sequence that is at least 45, 50, 55, 60, 65, 70, 75, 80, 85, 90, 95, 98, 99 or 100% identical to at least one of SEQ ID NO.'s: 8, 9, 10 and 11, or a nadA gene encoding such amino acid sequences.

Quinolinate phosphoribosyl-transferase (EC 2.4.2.19) is herein understood as an enzyme that catalyses the reaction: beta-nicotinate D-ribonucleotide+diphosphate+CO₂↔H pyridine-2,3-dicarboxylate+5-phospho-alpha-D-ribose 1-diphosphate (PRPP). The reaction is catalysed in the opposite direction. Quinolinate phosphoribosyl-transferase is also referred to as nicotinate-nucleotide diphosphorylase (carboxylating), quinolinate phosphoribosyltransferase (decarboxylating), quinolinic acid phosphoribosyltransferase, QAPRTase, NAD+ pyrophosphorylase, nicotinate mononucleotide pyrophosphorylase (carboxylating) and NadC and is the first NAD⁺ biosynthesis enzyme shared by both de novo NAD⁺ biosynthesis pathways from either tryptophan or aspartic acid. A quinolinate phosphoribosyl-transferase for use as heterologous NAD⁺ biosynthesis enzyme is further preferably defined as comprising an amino acid sequence that is at least 45, 50, 55, 60, 65, 70, 75, 80, 85, 90, 95, 98, 99 or 100% identical to at least one of SEQ ID NO.'s: 13, 14, 15 and 16, or a nadC gene encoding such amino acid sequences.

A preferred microbial host cell of the invention comprises at least a nucleotide sequence encoding a heterologous NAD⁺ biosynthesis enzyme that is a heterologous L-aspartate oxidase. More preferably a host cell of the invention comprises nucleotide sequences encoding two or all three of the heterologous NAD⁺ biosynthesis enzymes from the group consisting of L-aspartate oxidase, quinolinate synthase and quinolinate phosphoribosyl-transferase. Thus, a microbial host cell of the invention can comprise a single heterologous nadB, nadA or nadC gene, or the microbial host cell of the invention can comprise combinations of heterologous nadB and nadA genes, heterologous nadB and nadC genes, heterologous nadA and nadC genes or heterologous nadB, nadA and nadC genes.

In preferred embodiment of the invention, the microbial host cell comprises a genetic modification that reduces or eliminates the specific activity of at least one enzyme of an endogenous NAD⁺ biosynthesis pathway in the host cell. Preferably, in the host cell, the genetic modification reduces or eliminates the specific activity of an endogenous NAD⁺ biosynthesis enzyme that corresponds to (i.e. that catalyses the same reaction and/or has the same EC-number as) the heterologous NAD⁺ biosynthesis enzyme(s) encoded by the nucleotide sequence(s) comprised in the host cell. More preferably, in the host cell, the nucleotide sequence encoding a heterologous NAD⁺ biosynthesis enzyme replaces an endogenous nucleotide sequence encoding the corresponding endogenous NAD⁺ biosynthesis enzyme, whereby preferably all copies of an endogenous nucleotide sequence are replaced.

In an embodiment of the invention, the microbial host cell comprises an inducible promoter. In a preferred embodiment of the invention, the inducible promoter is present but not induced. It has been observed that by using an inducible promoter but not inducing it, the results as disclosed herein are the strongest. The natural leakiness of the promoter-system may leads to a “golden standard” on expression levels. Therefore, in one embodiment, the invention pertains to a microbial host cell comprising a nucleotide sequence encoding a heterologous NAD⁺ biosynthesis enzyme and an inducible promoter. In a further embodiment, the invention pertains to a microbial host cell comprising a nucleotide sequence encoding a heterologous NAD⁺ biosynthesis enzyme and an inducible promoter which is not induced. Examples of said inducible promoters include but are not limited to arabinose-inducible promoters, tetracycline-inducible promoters, lactose-inducible promoters, light inducible-promoters and temperature-inducible promoters.

A microbial host cell according to the invention can be a eukaryote or a prokaryote. In a preferred embodiment, the microbial host cell is a mesophilic microorganism. However, psychrophilic, psychrotrophic and/or thermophilic microorganism are explicitly not excluded as microbial host cells of the invention.

Thus, in one embodiment of the invention, the microbial host cell according to the invention is a eukaryotic microbial host cell, such as e.g. a fungal host cell. A preferred fungal host cell in accordance with the invention is a yeast or filamentous fungal host cell.

“Fungi” are herein defined as eukaryotic microorganisms and include all species of the subdivision Eumycotina (Alexopoulos, C. J., 1962, In: Introductory Mycology, John Wiley & Sons, Inc., New York). The terms “fungus” and “fungal” thus include or refers to both filamentous fungi and yeast.

“Filamentous fungi” are herein defined as eukaryotic microorganisms that include all filamentous forms of the subdivision Eumycotina and Oomycota (as defined in “Dictionary of The Fungi”, 10th edition, 2008, CABI, UK, www.cabi.org). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of the genera Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, and Ustilago.

Preferred filamentous fungal species as parent host cells for the invention belong to a species of an Aspergillus, Myceliophthora, Penicillium, Talaromyces or Trichoderma genus, and more preferably a species selected from Aspergillus niger, Aspergillus awamori, Aspergillus foetidus, Aspergillus sojae, Aspergillus fumigatus, Talaromyces emersonii, Aspergillus oryzae, Myceliophthora thermophila, Trichoderma reesei, Penicillium chrysogenum, Penicillium simplicissimum and Penicillium brasilianum. Suitable strains of these filamentous fungal species are available from depository institutions known per se to the skilled person.

“Yeasts” are herein defined as eukaryotic microorganisms and include all species of the subdivision Eumycotina (Yeasts: characteristics and identification, J. A. Barnett, R. W. Payne, D. Yarrow, 2000, 3rd ed., Cambridge University Press, Cambridge UK; and, The yeasts, a taxonomic study, CP. Kurtzman and J. W. Fell (eds) 1998, 4th ed., Elsevier Science Publ. B. V., Amsterdam, The Netherlands) that predominantly grow in unicellular form. Yeasts may either grow by budding of a unicellular thallus or may grow by fission of the organism. Preferred yeasts cells for use in the present invention belong to the genera Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, and Sporobolomyces. A parental yeast host cell can be a cell that is naturally capable of anaerobic fermentation, more preferably alcoholic fermentation and most preferably anaerobic alcoholic fermentation. More preferably yeasts from species such as Kluyveromyces lactis, Saccharomyces cerevisiae, Hansenula polymorpha (new name: Ogataea henricii), Yarrowia lipolytica, Candida tropicalis and Pichia pastoris (new name: Komagataella pastoris).

Preferably, a microbial host cell according to the invention is a prokaryote. The term “prokaryotic host cell” includes any microbial host cell, in which the genome is freely present within the cytoplasm (often as a circular structure), i.e. a cell, in which the genome is not surrounded by a nuclear membrane. A prokaryotic cell is further characterized in that it is not necessarily dependent on oxygen and its ribosomes are smaller than that of eukaryotic cells. Prokaryotic host cells according to the invention include archaebacteria and eubacteria. In dependence on the composition of the cell wall eubacteria can be divided into gram-positive bacteria, gram-negative bacteria and cyanobacteria, all of which are suitable as microbial host cell of the invention.

A preferred prokaryotic host cell according to the invention is a host cell of a genus selected from the group consisting of: Escherichia, Anabaena, Actinomyces, Acetobacter, Caulobacter, Clostridium, Gluconobacter, Gluconacetobacter, Rhodobacter, Pseudomonas, Paracoccus, Bacillus, Brevibacterium, Corynebacterium, Rhizobium, Sinorhizobium, Flavobacterium, Klebsiella, Enterobacter, Lactobacillus, Lactococcus, Streptococcus, Oenococcus, Leuconostoc, Pediococcus, Carnobacterium, Propionibacterium, Enterococcus, Bifidobacterium, Methylobacterium, Micrococcus, Staphylococcus, Streptomyces, Zymomonas, Streptococcus, Bacteroides, Selenomonas, Megasphaera, Burkholderia, Cupriavidus, Ralstonia, Methylobacterium, Methylovorus, Rhodopseudomonas, Acidiphilium, Dinoroseobacter, Agrobacterium, Sulfolobus or Sphingomonas. A further preferred prokaryotic host cell according to the invention is a host cell of a species selected from the group consisting of: Bacillus subtilis, Bacillus amyloliquefaciens, Bacillus licheniformis, Bacillus puntis, Bacillus megaterium, Bacillus halodurans, Bacillus pumilus, Gluconobacter oxydans, Caulobacter crescentus, Methylobacterium extorquens, Methylobacterium radiotolerans, Methylobacterium nodulans, Rhodobacter sphaeroides, Pseudomonas zeaxanthinifaciens, Pseudomonas putida, Pseudomonas putida S12, Paracoccus denitrificans, Escherichia coli, Corynebacterium glutamicum, Staphylococcus carnosus, Streptomyces lividans, Sinorhizobium meliloti, Bradyrhizobium japonicum, Rhizobium radiobacter, Rhizobium leguminosarum, Rhizobium leguminosarum bv. trifolii, Agrobacterium radiobacter, Cupriavidus basilensis, Cupriavidus necator (Ralstonia eutropha), Ralstonia pickettii, Burkholderia phytofirmans, Burkholderia phymatum, Burkholderia xenovorans, Burkholderia graminis, Rhodopseudomonas palustris, Acidiphilium cryptum, Dinoroseobacter shibae, Sulfolobus acidocaldarius, Sulfolobus islandicus, Sulfolobus solfataricus, and Sulfolobus tokodaii.

In a second aspect, the invention pertains to processes wherein the microbial host cells of the invention are used. Preferably the microbial host cells of the invention are employed in fermentation processes. In one embodiment, the invention relates to a process for producing a fermentation product, wherein preferably, the process comprises the steps of: (a) culturing a microbial host cell as defined herein above in a medium, whereby the host cell converts nutrients in the medium to the fermentation product; and, (b) optionally, recovery of the fermentation product. The fermentation may be carried out at conventionally used conditions, well known to the skilled person in the art, suitable for the fermenting organism in question. The process may be performed as a batch, fed-batch or as a continuous process. Preferred fermentation processes are anaerobic processes. A preferred process according to the invention for producing a fermentation product comprises a shift in temperature, wherein preferably the shift in temperature is a shift of at least 2, 5, 7 or 10° C.

A microbial cell according to the invention, wherein the microbial cell is not Escherichia Coli. A microbial cell according to the invention, wherein the microbial cell is not an Escherichia Coli cell that expresses NadA from Thermotoga maritima. A microbial cell according to the invention, wherein the microbial cell is not an Escherichia Coli cell that expresses NadB from Sulfolobud tokodaii. A microbial cell according to the invention, wherein the microbial cell is not an Escherichia Coli cell that expresses NadC from Thermotoga maritima. A microbial cell according to the invention, wherein the microbial cell is not an Escherichia Coli which expresses NadA from Thermotoga maritima, NadB from Sulfolobud tokodaii or NadC from Thermotoga maritima. A microbial cell according to the invention, wherein the microbial cell is not an Escherichia Coli which expresses NadA from Thermotoga maritima, NadB from Sulfolobud tokodaii or NadC from Thermotoga maritima at 37° C.

According to the invention the term “fermentation product” can be any substance derived from fermentation, i.e. a process including a fermentation step using a fermenting organism wherein a fermenting microbial host cell of the invention is cultured in a medium comprising nutrients that are converted by the host cell into the fermentation product. The fermentation product can be, without limitation, an alcohol (e.g. arabinitol, n-butanol, isobutanol, ethanol, glycerol, methanol, ethylene glycol, 1,3-propanediol [propylene glycol], butanediol, glycerin, sorbitol, and xylitol); an alkane (e.g. pentane, hexane, heptane, octane, nonane, decane, undecane, and dodecane), a cycloalkane (e.g. cyclopentane, cyclohexane, cycloheptane, and cyclooctane), an alkene (e.g. pentene, hexene, heptene, and octene); an amino acid (e.g. aspartic acid, glutamic acid, glycine, lysine, serine, and threonine); a gas (e.g. methane, hydrogen (H₂), carbon dioxide (CO₂), and carbon monoxide (CO)); isoprene; a ketone (e.g. acetone); an organic acid (e.g. acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); a polyketide; antibiotics (e.g. penicillin and tetracycline); enzymes; (pro)vitamins (e.g. riboflavin, B12, beta-carotene); and hormones. The fermentation product can also be biomass or protein as a high value product.

Subsequent to fermentation the fermentation product may be separated from the fermentation medium and/or from the fermenting microbial host cell. Methods for recovery of fermentation products are well known in the art.

In a third aspect, the invention relates to methods wherein an NAD⁺ biosynthesis enzyme, or more conveniently a nucleotide sequence encoding the NAD⁺ biosynthesis enzyme, is used to change at least one of the cardinal temperatures of a microbial host cell, and/or to improve the resistance of a microbial host cell to a shift in temperature. Preferably in this aspect, a nucleotide sequence encoding a NAD⁺ biosynthesis enzyme that is heterologous to a microbial host cell is used for at least one of: a) changing at least one of the minimum, maximum and optimum growth temperature of the microbial host cell; and, b) improving resistance of the microbial host cell to a shift in temperature. The method preferably comprises the step of introducing into the microbial host cell a nucleic acid construct for expression of the nucleotide sequence encoding the NAD⁺ biosynthesis enzyme that is heterologous to a microbial host cell. Preferably in this aspect, at least one of the microbial host cell and the nucleotide sequence encoding the NAD⁺ biosynthesis enzyme that is heterologous to the host cell is as defined herein above. In this embodiment of the invention, preferably, at least one of the minimum, maximum and optimum growth temperature of the microbial host cell is changed by at least 1, 2, 5, 10 or 20° C., more preferably at least the optimum growth temperature of the microbial host cell is changed by at least 1, 2, 5, 10 or 20° C. The minimum, maximum and optimum growth temperatures can be changed towards a lower temperature by at least 1, 2, 5, 10 or 20° C., but preferably at least one of the minimum, maximum and optimum growth temperature is changed towards a higher temperature by at least 1, 2, 5, 10 or 20° C. When improving microbial host cell's resistance to a shift in temperature, preferably, the lag phase of the microbial host cell upon a shift in temperature of at least 2, 5, 7 or 10° C., is reduced by at least a factor 1.1, 1.2, 1.5, 2, 5, 10 or 20. Preferably, the microbial host cell's resistance to a shift to a higher temperature is improved.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 0.1% of the value.

All patent and literature references cited in the present specification are hereby incorporated by reference in their entirety.

The present invention is further described by the following examples which should not be construed as limiting the scope of the invention.

### DESCRIPTION OF THE FIGURES

FIG. 1. Overview of the pathway for de novo biosynthesis of NAD⁺. L-Aspartate oxidase (NadB) and quinolinate synthase (NadA) are part of the quinolinate synthase system. Quinolinate is then processed with nicotinate by NadC (quinolinate phosphoribosyl-transferase) to form nicotinate mononucleotide.

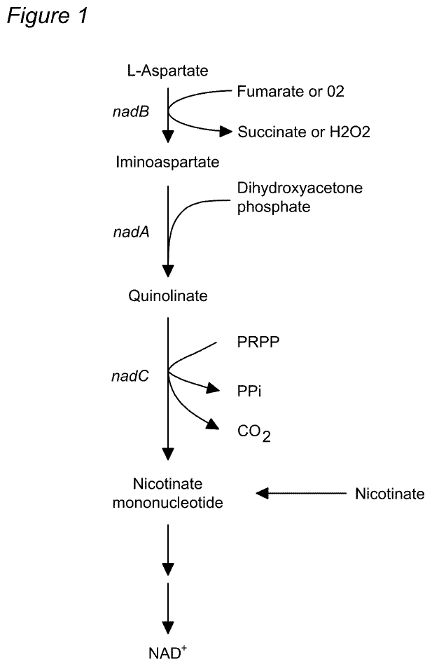

FIG. 2. E. coli growth curve after a temperature shift from 37 to 44° C. BW25113 wt, JW2558 (E. coli BW25113 ΔnadB) EnadB, and JW2558 (E. coli BW25113 ΔnadB) are used as controls. Growth monitored via a plate reader experiment, inoculated at OD₆₀₀ 0.05. Strains grown in M9 minimal medium. EnadB indicates expression of E. coli nadB from plasmid; PnadB indicates expression of P. putida nadB from plasmid; and BnadB indicates expression of B. smithii nadB from plasmid.

FIG. 3. Growth curves of P. putida KT2440 derivatives after a temperature shift from 30 to 40° C. P. putida KT2440 ΔnadB PnadB and P. putida KT2440 ΔnadB are used as controls. Growth monitored via a plate reader experiment, inoculated at OD₆₀₀ 0.05. Strains grown in DeBont minimal medium. KT2440 p638- indicates empty expression vector as negative control; KT2440 EnadB indicates expression of E. coli nadB from plasmid; KT2440 PnadB indicates expression of P. putida nadB from plasmid; and KT2440 TnadB indicates expression of T. flocculiformis nadB from plasmid.

FIG. 4. Flask growth experiments with P. putida KT2440 derivatives. Strains precultured in DeBont minimal medium o/n at 30° C., then passaged with a starting OD of 0.05 to 20 ml fresh medium. Growth was monitored by sampling at given time points and determining the OD, over a period of 21 h. P. putida KT2440 ΔnadB PnadB and P. putida KT2440 ΔnadB are used as controls. KT2440 p638- indicates empty expression vector as negative control; KT2440 EnadB indicates expression of E. coli nadB from plasmid; KT2440 PnadB indicates expression of P. putida nadB from plasmid; KT2440 BnadB indicates expression of B. smithii nadB from plasmid; and KT2440 TnadB indicates expression of T. flocculiformis nadB from plasmid.

FIG. 5. Flask growth experiments with P. putida KT2440 derivatives. Strains were isolated from the 40° C. cultures and passaged with a starting OD of 0.05 to 20 ml fresh medium. The cultures were grown at 41° C. Growth was monitored by sampling at given time points and determining the OD, over a period of 21 h. P. putida KT2440 ΔnadB PnadB and P. putida KT2440 ΔnadB are used as controls. KT2440 p638- indicates empty expression vector as negative control; KT2440 p638 EnadB indicates expression of E. coli nadB from plasmid; KT2440 p638 PnadB indicates expression of P. putida nadB from plasmid; KT2440 p638 BnadB indicates expression of B. smithii nadB from plasmid; and KT2440 p638 TnadB indicates expression of T. flocculiformis nadB from plasmid.

FIG. 6. Top: Saccharomyces cerevisiae at 30° C., Bottom Saccharomyces cerevisiae at 41° C. From left to right YNB medium, YNB+Aspartate medium and YNB+Tryptophan medium. Depicted are S. cerevisiae BY4741 WT (triangle), pRV156 empty plasmid (circle), pRV156 nadABC B. smithii (square), pRV157 nadABC P. putida (diamond). Growth in flasks was monitored for 24 hours. Blank flasks with un-inoculated medium were used to correct for background emission.

### EXAMPLES

Materials and Methods

Strains and Culture Conditions

Bacterial strains and plasmids used in this study are listed in Table 1. E. coli DH5a was used for routine cloning procedures and plasmid maintenance, and was routinely cultivated at 37° C. in aerated conditions in LB medium (10 g/l tryptone, 10 g/l NaCl and 5 g/l yeast extract, adding 15 g/l agar for solid medium), optionally containing antibiotics for plasmid selection (10 μg/ml gentamycin as indicated). P. putida KT2440, P. putida KT2440 ΔnadB, E. coli BW25113 or E. coli BW25113 ΔnadB (JW2558) were routinely cultivated under oxic conditions in minimal medium. P. putida was cultured at 30° C. in De Bont minimal medium (DB) [10] (3.88 g/L K₂HPO₄, 1.63 g/l NaH₂PO₄.2H₂O, 2.00 g/l (NH₄)₂SO₄, 0.1 g/l MgCl₂.6H₂O, 10 mg/l EDTA, 2 mg/l ZnSO₄.7H₂O, 1 mg/l CaCl₂.2H₂O, 5 mg/l FeSO₄.7H₂O, 0.2 mg/l Na₂MoO₄.2H₂O, 0.2 mg/l CuSO₄.5H₂O, 0.4 mg/l CoCl₂.6H₂O, 1 mg/l MnCl₂.2H₂O, All chemicals and antibiotics were purchased at Machery-Nagel GmbH & Co. (Düren). E. coli was cultured at 37° C. in M9 minimal medium (5×M9 minimal salts, 1M MgSO₄, 1M CaCl₂, Thiamin and 100× DeBont Trace Elements). In all experiments 20 g/l glucose was used as the sole carbon source, with 10 μg/ml gentamycin to select for recombinant strains. The optical cell density was analysed photometrically at 600 nm (OD600). Precultures were prepared by overnight (o/n) cultivation at 200 rpm at the indicated cultivation temperature.

Plasmid Construction

DNA segments were amplified by colony PCR using the Phire Green Hot Start II DNA Polymerase kit (Thermo Fisher Scientific, Waltham, Mass., USA), according to the manufacturer's protocol. Clones were regularly checked by colony PCR and sequencing. All primer oligonucleotides used were purchased from Sigma-Aldrich Co. (Table 2). Restriction enzymes were obtained from NEB (New England BioLabs®_{inc.}). Using the Standardized SEVA plasmid system [17, 18], the cargo (nadB from E. coli, P. putida, T. flocculiformis or B. smithii) was designed with BamHI and EcoRI restriction sites on the 5′-end and 3′-end, respectively. DNA fragments were purified from agarose gel using the Machery-Nagel GmbH & Co. KG Gel Purification Kit (Machery-Nagel GmbH & Co. Düren, Germany). Plasmid inserts were verified by gel electrophoresis or DNA sequencing via Lightrun sequencing at GATC Biotech. T4 DNA Ligase (Roche Applied Science Indianapolis, Ind. USA) was used to ligate the isolated DNA fragments in the pSEVA 638 backbone. DNA segments were stored at −20° C. Plasmids were electroporated into competent P. putida KT2440 ΔnadB or into competent E. coli JW2558.

Growth Experiments

Platereader experiments were performed to monitor growth at varying temperatures closely over periods of 24-72 h. Recombinant E. coli was precultured at 37° C. in minimal M9 medium with glucose and 10 μg/ml Gentamycin as a selection marker. Recombinant P. putida was precultured at 30° C. in minimal DeBont medium with 20 g/l glucose and 10 μg/ml Gentamycin as a selection marker. 96 wells-plates were inoculated at a starting OD of 0.05. As a control, blank wells and wells inoculated with wild-type E. coli BW25113 or P. putida KT2440 were prepared with M9 or DeBont medium without selection marker. The platereader was run 48 to 72 h at varying temperatures while measuring the OD every 20 minutes, whilst shaking continuously. The plates were taped on both sides to counter condensation at higher temperatures.

Statistical Analysis

All of the reported experiments were independently repeated twice. Figures represent the mean values of corresponding biological triplicates and the standard deviation. The level of significance of the differences when comparing results was evaluated by means of analysis of variance (ANOVA), with α=0.05.

### Example 1

We found that if the gene coding for nadB in a mesophilic strain is replaced by the nadB gene of a thermophile, the mesophilic strain is more resistant to shifts in temperature instantly. Since nadB deletion directly influences the redox balance, changing either nadC or nadA has a similar effect. The increase in tolerance also occurs when shifting to lower temperatures during growth, after the integration of a psychrophilic nadB, nadC or nadA gene.

This finding was proved by using two mesophilic strains, E. coli BW25113 and P. putida KT2440, both from which the nadB gene was removed (E. coli ΔnadB and P. putida ΔnadB). The nadB gene was reintroduced via the pSEVA plasmid system. Plasmids were prepared with the nadB gene of E. coli BW25113, P. putida KT2440, B. smithii DSM 4216 (a thermophile, optimal growth temperature of 55° C., temperature growth range of 25-65° C.) and T. flocculiformis DSM2094 (a psychrotolerant mesophile, optimal growth temperature of 35° C., temperature growth range of 2° C.-40° C.). The nadB knock out strains were used as negative controls. All plasmids were introduced in either the E. coli ΔnadB strain or the P. putida ΔnadB strain. The strains were cultivated in rich LB or minimal M9 medium. Precultures were prepared at the strain specific optimal temperature (P. putida at 30° C., E. coli at 37° C.). Growth experiments were performed in a plate reader to determine the lag phase before adjusting to a temperature shift.

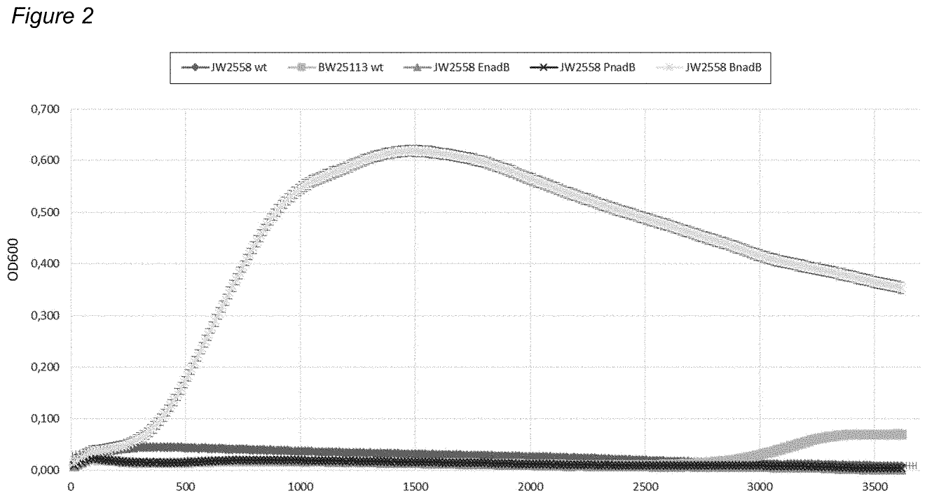

FIG. 2 shows that in E. coli, the replacement of the inherent gene by the thermophilic B. smithii nadB gene results in a 10 times shorter lag phase when shifting the growth temperature from 37° C. to 44° C., compared to a wild type control.

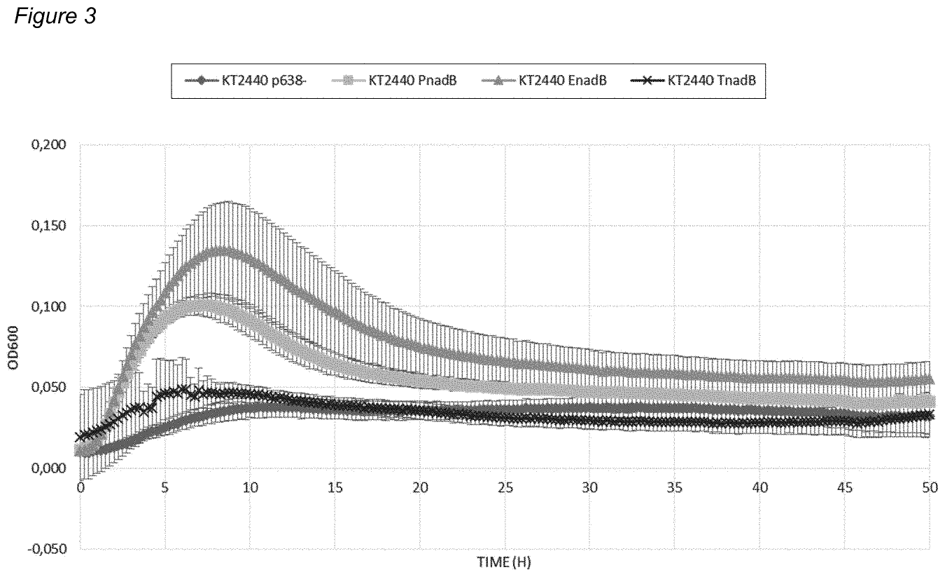

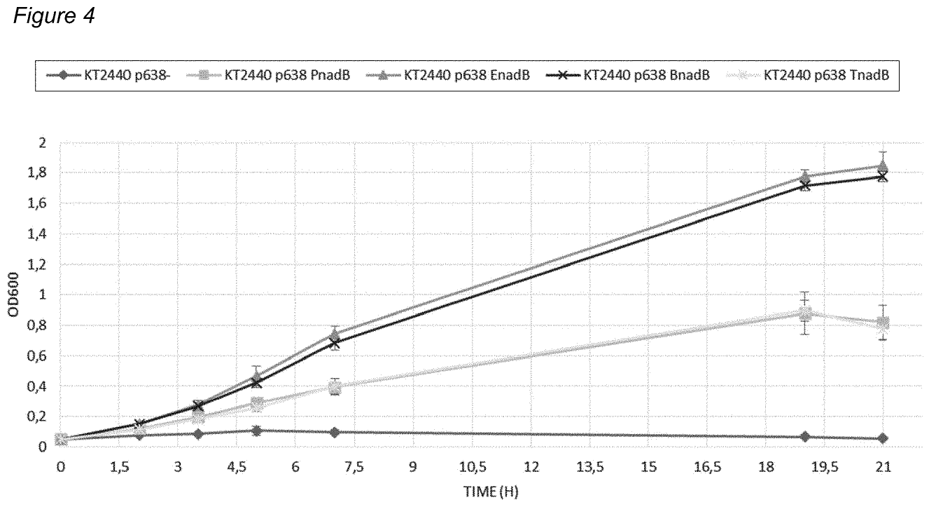

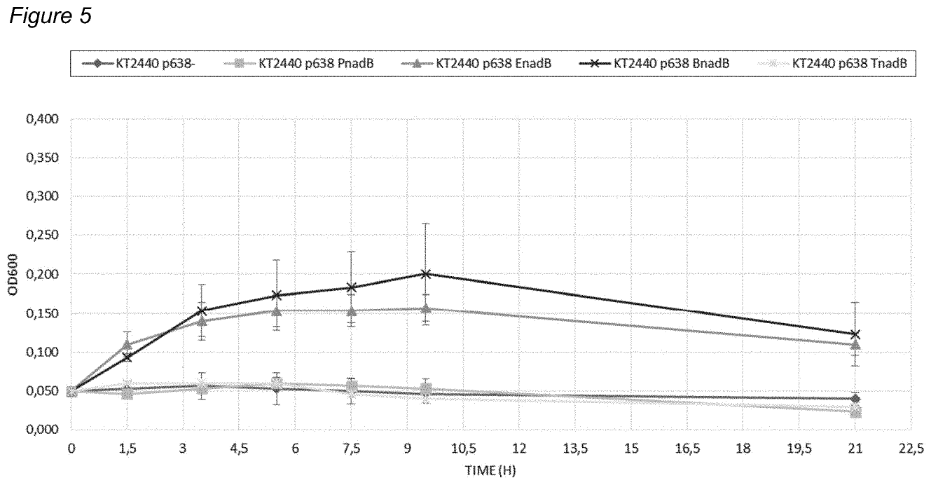

FIG. 3 shows that in P. putida, the replacement of the inherent gene by that of E. coli or B. smithii caused the maximum growth rate to increase by a factor 2, from 0.0453 to 0.0943 h⁻¹ (E. coli) or 0.0922 h⁻¹ (B. smithii) after a temperature shift from 30° C. to 40° C. FIG. 4 shows the growth of the various P. putida strains in shake flasks at 40° C. Isolation of the growing strains and increasing the temperature by 1° C. even resulted in growth at 41° C., which P. putida normally is not capable of (FIG. 5).

### Example 2

Further experimentation including the expression of nadA-nadB-nadC of P. putida or B. smithii in S. cerevisiae (BY4741: MATa his3Δ1 leu2Δ0 met15Δ0 ura3Δ0)

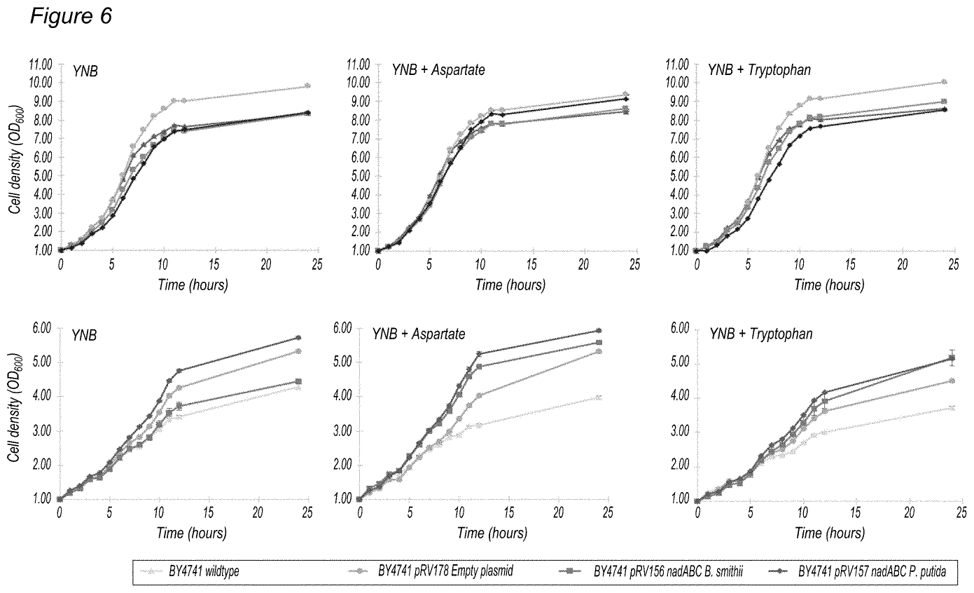

FIG. 6 shows that expression of nadA-nadB-nadC of P. putida or B. smithii in S. cerevisiae consistently has a positive effect on temperature tolerance. The increased temperature tolerance effect also does not hold under anoxic conditions, linking the surplus of NAD+ to a decrease in reactive oxygen species which is now shown to increase temperature tolerance.

TABLE 1
Bacterial strains and plasmids used in this study

| | | Growth | |
|---|---|---|---|
| Bacterial strain or | | temperature | Source or |
| plasmid | Relevant characteristics ^{a} | range | references |
| Escherichia coli | | | |
| DH5α | Cloning host; ϕ80lacZΔM15 recA1 endA1 | 27-44° C. | [11] |
| | gyrA96 thi-1 hsdR17(r_{κ}⁻ m_{κ}⁺ supE44 relA1 | | |
| | deoR Δ(lacZYA-argF)U169 | | |
| BW25113 | lacI^{q} rrnB_{T14} ΔlacZ_{WJ16} hsdR514 | | [12]-[14] |
| | ΔaraBAD_{AH33} ΔrhaBAD_{LD78} | | |
| JW2558 | BW25113 with ΔnadB | | [12] |
| Pseudomonas | | | |
| putida | | | |
| KT2440 | Wild-type strain, spontaneous restriction- | 25-40° C. | [15] |
| | deficient derivative of strain mt-2 cured of | | |
| | the TOL-plasmid pWW0 | | |
| KT2440 ΔnadB | KT2440 with a knocked out nadB gene | | This study |
| Bacillus smithii | | | |
| DSM 4216 | Wild type strain | 25-65° C. | [16] |
| Trichococcus | | | |
| flocculiformis | | | |
| DSM2094 | Wild type strain | | [19] |
| Plasmid | | | |
| pSEVA 638^{b} | Expression vector; pBBR1 xylS-Pm Gm^{R} | | [17, 18] |
| pSEVA 638 | pSEVA 638 with nadB from Escherichia coli | | This study |
| N_Eco^{b} | | | |
| pSEVA 638 N | pSEVA 638 with nadB from Pseudomonas | | This study |
| Ppu^{b} | putida | | |
| pSEVA 638 N | pSEVA 638 with nadB from Bacillus smithii | | This study |
| Bsm^{b} | | | |
| pSEVA 638 N Tfl^{b} | pSEVA 638 with nadB from T. flocculiformis | | This study |

^{a} Antibiotic marker: Gm, gentamycin
^{b}Plasmids belonging to the SEVA (Standard European Vector Architecture) collection [17, 18].

TABLE 2
Primers used in this study

| Primer purpose | Forward primer | Reverse primer |
|---|---|---|
| E. coli BW25113 | ACTGGAGCTCCACCCCAGGAa | ACTGGGATCCTTATCTGTTTATGTA |
| nadB isolation | ggaggaaaaaacatATGAATACTCT | ATGATTGCCGG |
| | CCCTGAACATTC | |
| P. putida | ACTGGAGCTCCACCCCAGGAa | ACTGGGATCCTCAGAGCGGGTTAA |
| KT2440 nadB | ggaggaaaaaacatATGAGCCAACA | GGATGGTG |
| isolation | ATTCCAACATGATGTCC | |
| B. smithii DSM | cagtCATatggagaaagaagcggatg | cagtGGATCCttaagcatggattccagtttg |
| 4216 nadB | | |
| isolation | | |
| T. flocculiformis | CATATGATGCGCAACTATGATG | GGATCCTTACTTTGCATGAGCTTCC |
| DSM 2094 nadB | TCC | TC |
| isolation | | |

TABLE 3
NAD⁺ biosynthesis genes and enzymes in the sequence listing.

| Enzyme/gene | Type | SEQ ID NO |
|---|---|---|
| B. smithii nadB | DNA | 1 |
| B. smithii nadB | protein | 2 |
| T. flocculiformis nadB | DNA | 3 |
| T. flocculiformis nadB | protein | 4 |
| P. putida KT2440 nadB | protein | 5 |
| E. coli BW25113 nadB | protein | 6 |
| B. smithii nadA | DNA | 7 |
| B. smithii nadA | protein | 8 |
| T. flocculiformis nadA | protein | 9 |
| P. putida KT2440 nadA | protein | 10 |
| E. coli BW25113 nadA | protein | 11 |
| B. smithii nadC | DNA | 12 |
| B. smithii nadC | protein | 13 |
| T. flocculiformis nadC | protein | 14 |
| P. putida KT2440 nadC | protein | 15 |
| E. coli BW25113 nadC | protein | 16 |

### REFERENCES

[1] K. N. Thomas, A. Ben-Bassat, and J. G. Zeikus, “Ethanol Production by Thermophilic Bacteria: Fermentation of Cellulosic Substrates by Cocultures of Clostridium thermocellum and Clostridium thermohydrosulfuricum,” Appl. Environ. Microbiol., vol. 41, no. 6, pp. 1337-1343, January 1981.
[2] J. G. Zeikus, “Thermophilic bacteria: Ecology, physiology and technology,” Enzyme Microb. Technol., vol. 1, no. 4, pp. 243-252, October 1979.
[3] H. Sakuraba, K. Yoneda, I. Asai, H. Tsuge, N. Katunuma, and T. Ohshima, “Structure of 1-aspartate oxidase from the hyperthermophilic archaeon Sulfolobus tokodaii,” Biochim. Biophys. Acta, vol. 1784, no. 3, pp. 563-571, March 2008.
[4] I. Marinoni et al., “Characterization of 1-aspartate oxidase and quinolinate synthase from Bacillus subtilis,” FEBS J., vol. 275, no. 20, pp. 5090-5107, October 2008.
[5] S. Nasu, F. D. Wicks, and R. K. Gholson, “L-Aspartate oxidase, a newly discovered enzyme of Escherichia coli, is the B protein of quinolinate synthetase.,” J. Biol. Chem., vol. 257, no. 2, pp. 626-632, January 1982.
[6] W. T. Booth e.a., “Streptococcus pyogenes quinolinate-salvage pathway-structural and functional studies of quinolinate phosphoribosyl transferase and NH3-dependent NAD+ synthetase”, The FEBS Journal, vol. 284, nr. 15, pp. 2425-2441, aug. 2017.
[7] P. De Maayer, D. Anderson, C. Cary, en D. A. Cowan, “Some like it cold: understanding the survival strategies of psychrophiles”, EMBO Rep, vol. 15, nr. 5, pp. 508-517, mei 2014.
[8] M. Rafiq e.a., “Recovery of metallo-tolerant and antibiotic resistant psychrophilic bacteria from Siachen glacier, Pakistan”, PLoS ONE, vol. 12, nr. 7, p. e0178180, 2017.
[9] P. Poltronieri, Microbiology in Dairy Processing: Challenges and Opportunities. John Wiley & Sons, 2017.
[10] S. Hartmans, J. P. Smits, M. J. van der Werf, F. Volkering, and J. A. M. de Bont, “Metabolism of Styrene Oxide and 2-Phenylethanol in the Styrene-Degrading Xanthobacter Strain 124X,” Appl. Environ. Microbiol., vol. 55, no. 11, pp. 2850-2855, November 1989.
[11] D. Hanahan and M. Meselson, “Plasmid screening at high colony density,” Gene, vol. 10, no. 1, pp. 63-67, June 1980.
[12] T. Baba et al., “Construction of Escherichia coli K-12 in-frame, single-gene knockout mutants: the Keio collection,” Mol. Syst. Biol., vol. 2, p. 2006.0008, February 2006.
[13] F. Grenier, D. Matteau, V. Baby, and S. Rodrigue, “Complete Genome Sequence of Escherichia coli BW25113,” Genome Announc., vol. 2, no. 5, pp. e01038-14, October 2014.
[14] K. A. Datsenko and B. L. Wanner, “One-step inactivation of chromosomal genes in Escherichia coli K-12 using PCR products,” Proc. Natl. Acad. Sci. U.S.A, vol. 97, no. 12, pp. 6640-6645, June 2000.
[15] M. Bagdasarian et al., “Specific-purpose plasmid cloning vectors II. Broad host range, high copy number, RSF 1010-derived vectors, and a host-vector system for gene cloning in Pseudomonas,” Gene, vol. 16, no. 1-3, pp. 237-247, December 1981.
[16] L. K. NAKAMURA, I. BLUMENSTOCK, and D. CLAUS, “Taxonomic Study of Bacillus coagulans Hammer 1915 with a Proposal for Bacillus smithii sp. nov.,” Int. J. Syst. Evol. Microbiol., vol. 38, no. 1, pp. 63-73, 1988.
[17] R. Silva-Rocha et al., “The Standard European Vector Architecture (SEVA): a coherent platform for the analysis and deployment of complex prokaryotic phenotypes,” Nucleic Acids Res., vol. 41, no. Database issue, pp. D666-D675, January 2013.
[18] E. Martinez-Garcia, T. Aparicio, A. Goñi-Moreno, S. Fraile, and V. de Lorenzo, “SEVA 2.0: an update of the Standard European Vector Architecture for de-/re-construction of bacterial functionalities,” Nucleic Acids Res., vol. 43, no. Database issue, pp. D1183-D1189, January 2015.
[19] Strepis et al., Int J Syst Evol Microbiol. 2016 October; 66(10):3957-3963. doi: 10.1099/ijsem.0.001294. Epub 2016 Jul. 11.

1. A microbial host cell comprising a nucleotide sequence encoding a heterologous NAD+ biosynthesis enzyme, wherein at least one of:
a) the heterologous NAD+ biosynthesis enzyme is from a microbial donor organism with an optimum growth temperature that is different from the optimum growth temperature of the microbial host cell, or from a microbial donor organism that has a wider range of growth temperatures than the microbial host cell; and,
b) the heterologous NAD+ biosynthesis enzyme has a higher activity than the corresponding endogenous NAD+ biosynthesis enzyme of the host cell at a temperature that differs from the optimum growth temperature of the host cell, as determined in an assay for activity of the NAD+ biosynthesis enzyme wherein the activity of the endogenous and heterologous NAD+ biosynthesis enzymes is determined over a period of time of at least 10 minutes.

2. A microbial host cell according to claim 1, wherein the heterologous NAD+ biosynthesis enzyme is selected from the group consisting of L-aspartate oxidase, quinolinate synthase and quinolinate phosphoribosyl-transferase, and wherein preferably the microbial host cell comprises nucleotide sequences encoding two or all three of the heterologous NAD+ biosynthesis enzyme from the group consisting of L-aspartate oxidase, quinolinate synthase and quinolinate phosphoribosyl-transferase.

3. A microbial host cell according to claim 1 or 2, wherein the temperature difference in at least one of a) and b) is at least 2° C.

4. A microbial host cell according to any one of the preceding claim 1, wherein at least one of:
a) the heterologous NAD+ biosynthesis enzyme has a higher activity than the corresponding endogenous NAD+ biosynthesis enzyme in the host cell at a temperature that is higher than the optimum growth temperature of the host cell; and,
b) the heterologous NAD+ biosynthesis enzyme is from a microbial donor organism with an optimum growth temperature that is higher than the optimum growth temperature of the microbial host cell.

5. A microbial host cell according to claim 1 wherein the host cell comprises a genetic modification that reduces or eliminates the specific activity of an endogenous NAD+ biosynthesis enzyme that corresponds to the heterologous NAD+ biosynthesis enzyme encoded by the nucleotide sequence comprised in the host cell, wherein preferably, the nucleotide sequence encoding a heterologous NAD+ biosynthesis enzyme replaces the endogenous nucleotide sequence encoding the corresponding endogenous NAD+ biosynthesis enzyme.

6. A microbial host cell according to claim 1, wherein the host cell is a yeast, a filamentous fungus, a eubacterium or an archaebacterium, preferably a Gram-positive or a Gram-negative bacterium.

7. A microbial host cell according to claim 6, wherein the host cell is of a genus selected from the group consisting of: Escherichia, Anabaena, Actinomyces, Acetobacter, Caulobacter, Clostridium, Gluconobacter, Gluconacetobacter, Rhodobacter, Pseudomonas, Paracoccus, Bacillus, Brevibacterium, Corynebacterium, Rhizobium Sinorhizobium, Flavobacterium, Klebsiella, Enterobacter, Lactobacillus, Lactococcus, Streptococcus, Oenococcus, Leuconostoc, Pediococcus, Carnobacterium, Propionibacterium, Enterococcus, Bifidobacterium, Methylobacterium, Micrococcus, Staphylococcus, Streptomyces. Zymomonas, Streptococcus, Bacteroides, Selenomonas, Megasphaera, Burkholderia, Cupriavidus, Ralstonia, Methylobacterium, Methylovorus, Rhodopseudomonas, Acidiphilium, Dinoroseobacter, Agrobacterium, Sulfolobus, Sphingomonas, Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, Trichoderma, Ustilago, Saccharomyces, Kluyveromyces, Candida, Pichia, Schizosaccharomyces, Hansenula, Kloeckera, Schwanniomyces, Yarrowia, Cryptococcus, Debaromyces, Saccharomycecopsis, Saccharomycodes, Wickerhamia, Debayomyces, Hanseniaspora, Ogataea, Kuraishia, Komagataella, Metschnikowia, Williopsis, Nakazawaea, Torulaspora, Bullera, Rhodotorula, and Sporobolomyces.

8. A microbial host cell according to claim 1, wherein the microbial donor organism is a psychrophilic, a psychrotrophic or a thermophilic organism and wherein preferably the microbial host cell is a mesophile.

9. A microbial host cell according to claim 1, wherein the heterologous NAD+ biosynthesis enzyme is a modified version of an enzyme that is endogenous to the host cell, which modified version enzyme comprises at least one modification in its amino acid sequence as compared to the endogenous enzyme, and wherein the modified version has a higher activity than the endogenous enzyme at a temperature that differs from the optimum growth temperature of the host cell, in an assay for activity of the NAD+ biosynthesis enzyme wherein the activity of the endogenous and the modified enzymes is determined over a period of time of at least 10 minutes.

10. A microbial host cell according to any one of the preceding claim 1, wherein the heterologous NAD+ biosynthesis enzyme comprises an amino acid sequence selected from the group consisting of:
a) an amino acid sequence that is at least 45% identical to SEQ ID NO: 2; b) an amino acid sequence that is at least 45% identical to SEQ ID NO: 4; c) an amino acid sequence that is at least 45% identical to SEQ ID NO: 5; d) an amino acid sequence that is at least 45% identical to SEQ ID NO: 6; e) an amino acid sequence that is at least 45% identical to SEQ ID NO: 8; f) an amino acid sequence that is at least 45% identical to SEQ ID NO: 9; g) an amino acid sequence that is at least 45% identical to SEQ ID NO: 10; h) an amino acid sequence that is at least 45% identical to SEQ ID NO: 11; i) an amino acid sequence that is at least 45% identical to SEQ ID NO: 13; j) an amino acid sequence that is at least 45% identical to SEQ ID NO: 14; k) an amino acid sequence that is at least 45% identical to SEQ ID NO: 15; and, 1) an amino acid sequence that is at least 45% identical to SEQ ID NO: 16.

11. A process for producing a fermentation product, the process comprises the steps of:
(a) culturing a host cell as defined in claim 1 in a medium, whereby the host cell converts nutrients in the medium to the fermentation product; and,
(b) optionally, recovery of the fermentation product.

12. A process according to claim 11, wherein the process comprises a shift in temperature, wherein preferably the shift in temperature is a shift of at least 2, 5, 7 or 10° C.

13. Use of a nucleotide sequence encoding a NAD+ biosynthesis enzyme that is heterologous to a microbial host cell, for at least one of:
a) changing at least one of the minimum, maximum and optimum growth temperature of the microbial host cell; and,
b) improving resistance of the microbial host cell to a shift in temperature, wherein preferably the resistance of the microbial host cell to a shift to a higher temperature is improved.

14. A use according to claim 13, wherein the microbial host cell comprises a nucleotide sequence encoding a heterologous NAD+ biosynthesis enzyme wherein at least one of:
a) the heterologous NAD+ biosynthesis enzyme is from a microbial donor organism with an optimum growth temperature that is different from the optimum growth temperature of the microbial host cell, or from a microbial donor organism that has a wider range of growth temperatures than the microbial host cell; and,
b) the heterologous NAD+ biosynthesis enzyme has a higher activity than the corresponding endogenous NAD+ biosynthesis enzyme of the host cell at a temperature that differs from the optimum growth temperature of the host cell, as determined in an assay for activity of the NAD+ biosynthesis enzyme wherein the activity of the endogenous and heterologous NAD+ biosynthesis enzymes is determined over a period of time of at least 10 minutes.

15. A use according to claim 13, wherein at least one of:
a) at least one of the minimum, maximum and optimum growth temperature of the microbial host cell is changed by at least 1° C.; and,
b) the lag phase of the microbial host cell upon a shift in temperature of at least 2° C., is reduced by at least a factor 1.1.
